(19) United States
(12) Patent Application Publication
(10) Pub. No.: US 2011/0160566 A1
(43) Pub. Date: Jun. 30, 2011

(54) MRI AND ULTRASOUND GUIDED TREATMENT ON A PATIENT

(76) Inventors: Labros Petropoulos, Winnipeg (CA); John K. Saunders, Winnipeg (CA)

(21) Appl. No.: 12/972,726

(22) Filed: Dec. 20, 2010

Related U.S. Application Data
(60) Provisional application No. 61/290,070, filed on Dec. 24, 2009.

(51) Int. Cl. A61B 5/055

(52) U.S. Cl. 600/411

(57) ABSTRACT

A method is provided for guiding a procedure on a region of interest in a body part of patient, where the body part moves within the patient by breathing, cardiac or other action. The procedure includes radiation therapy, or guidance of a probe for example for biopsy or brachytherapy. The method includes using an MRI system to obtain an MR image of the body part, during the procedure on the body part of the patient, obtaining real time ultrasound images of the body part of the patient as the body part moves within the body of the patient, registering the MR image of the body part with the ultrasound image of the body part so as to locate the region of interest in the ultrasound image of the body part and using the registered images to target the action to the region of interest as the body part moves.

This application claims the benefit under 35 U.S.C. 119 of the filing date of Provisional Application Ser. No.: 61/290,070 filed Dec. 24, 2009.

This invention relates to MRI and ultrasound guided treatment on a patient.

### BACKGROUND OF THE INVENTION

A radio therapy device generally includes a linear electron beam accelerator which is mounted on a gantry and which can rotate about an axis which is generally parallel to the patient lying on the patient couch. During the radiation therapy, the patient is treated using either an electron beam or an X-ray beam produced from the original electron beam. The electron or X-ray beam is focused at a target volume in the patient by the combination of the use of a collimator and the rotation of the source around the patient. The patient is placed on a couch which can be positioned such that the target lesion can be located in the plane of the electron beam as the gantry rotates in two directions.

The objective of the radiation therapy is to target the lesion with a high dose of radiation over time and to have minimal impact on all the surrounding normal tissue. The first task is to precisely locate the tumor in three-dimensional space. The best technique for this is MRI since this technology provides high resolution in the imaging of soft tissue to provide high soft tissue contrast.

Even though MRI provides good location of the tumor at the time of the measurement, these images are normally recorded two to three days prior to the treatment and so may not be completely representative of tumor location on the day of treatment. The oncologists therefore tend to increase the target volume to be certain that all of the tumor tissue receives the required dose of the radiation, even though this increase in the volume of the tissue exposed to radiation also necessarily targets healthy tissue with consequential damage to the healthy tissue. The expectation is that all cells in the targeted region will be killed and this includes both the lesion and the healthy tissue. This produces collateral damage and may have a significant impact of the quality of life of the patient.

Brachytherapy, also known as internal radiotherapy, sealed source radiotherapy, curietherapy or endocurietherapy, is a form of radiotherapy where a radiation source is placed inside or next to the area requiring treatment. Brachytherapy is commonly used as an effective treatment for many cancers and can also be used to treat tumors in many body sites.

In contrast to external radiation therapy, in which high-energy X-rays are directed at the tumor from outside the body, brachytherapy involves the precise placement of radiation sources directly at the site of the cancerous tumor. A key feature of brachytherapy is that the irradiation only affects a very localized area around the radiation sources. Exposure to radiation of healthy tissues further away from the sources is therefore reduced. In addition, if the patient moves or if there is any movement of the tumor within the body during treatment, the radiation sources retain their correct position in relation to the tumor. These characteristics of brachytherapy provide advantages over external therapy, the tumor can be treated with very high doses of localized radiation, whilst reducing the probability of unnecessary damage to surrounding healthy tissues.

However the accurate placement of the probe acting to locate the source or sources of radiation is of course essential to the effectiveness of the procedure.

An additional challenge to effective radiation treatment and other treatment actions is the effect of motion of the tumor or other region of interest in the body due to respiratory motion, cardiac motion, peristalsis, digestive system actions and other bodily functions. This can result in significant movement of the adjacent body parts so that tumor masses can move making the continuous accurate targeting for treatment difficult. Again therefore the oncologists generally increase the size of the target volume radiated to accommodate movement of the lesion during respiratory and cardiac movement.

In addition to the above issues in relation to external radiation therapies, breathing, cardiac, or other unwanted motion also poses a problem during the MR interventional, biopsy and brachytherapy procedures where a probe acting to effect the action required must be accurately located. Once again, MRI is viewed as the best modality for characterizing tumors on soft moving tissue due to the contrast resolution that offers. However, during a biopsy procedure utilizing MRI, a biopsy device is inserted into a targeted suspicious area of the soft organ, the patient is inserted inside the MRI scanner for verifying and then the patient again is removed from the magnet to complete the biopsy procedure. Thus, during the insertion of the biopsy device, the soft organ can be moved and thus the biopsy device will miss the targeted area. In this case, MRI will detect that the biopsy device is located at the correct spot and then, the biopsy device has to be readjusted, the patient inserted to the MR scanner once again for a verification before a biopsy can occur. Over the years physicians, because of their experience or by trial and error, have learned how to cope with performing biopsy procedures for moving organs. Although the MRI procedure is considered a real time procedure, it is actually not since the steps are taken separately and sequentially. That is, when the biopsy device is inserted inside the organ, the path of the device is not monitored by the MRI in real time. Only after an insertion is complete, is an MR image taken to verify the final position.

The error of positioning these devices is accelerated during a brachytherapy procedure, such as in the prostate where 12 to 18 tubes have to be inserted in the prostate for a treatment depending on the size and location of the lesion. The physician can perform the insertion of these tubes under MRI guidance but again no real time monitoring of the insertion of the tubes into the area to be treated is available. If the positions of the tubes are not located in the right area, the tubes must be repositioned and only after their repositioning, an MR image is obtained to verify their spatial accuracy relative to the target area for treatment. Once again, no real time navigation and tracking motion is available during the insertion of the brachytherapy tubes in the targeted treatment area.

In general major MR modalities like magnetic resonance imaging are usually incompatible with other imaging modalities like CT, X-ray, PET, SPECT and LINACs which are utilized for radiation therapy treatment. Unfortunately due to the ferromagnetic materials that are part of a LINAC, it can not coexist at the same space as the MR system due to the safety concerns. Thus for a radiation treatment on a patient moving soft organs, like the prostate, liver, pancreas, etc, a real time dynamic (Cine) image of the movement of the soft tissue during the breathing cycle is obtained. During the planning session, a broader area of radiation treatment has to be modeled, taking into account the movement of the patient's soft organs during a breathing motion and the uncertainty of time of delivering the radiation dose during the breathing cycle and the position of the tumor on the soft organ at that time.

A system has been developed by Philips in conjunction with Electra which shows that a LINAC can be used in combination with an MRI. Thus the two can co-exist but this may not be the ideal solution since there are many restrictions if a combined system is developed.

A number of attempts have been made to improve the accuracy of the location of the lesion for radiotherapy.

U.S. Pat. No. 7,494,467 (Makin) issued Feb. 24, 2009, involves an ultrasound transducer with an RF electrode for RF ablation procedures.

U.S. Pat. No. 5,178,146 (Giese) issued Jan. 12, 1993 discloses a grid system of contrast material which is compatible with MRI which is used to plan radiotherapy.

The following patents disclose a technique for identifying the target volume using MRI which is used to plan radiotherapy:

U.S. Pat. No. 5,402,783 (Friedman) assigned to Eco-Safe and issued Apr. 4, 1995;

U.S. Pat. No. 5,537,452 (Shepherd) issued Jul. 16, 1996;

U.S. Pat. No. 5,800,353 (McLaurin) issued Sep. 1, 1998;

U.S. Pat. No. 6,198,957 (Green) assigned to Varian and issued Mar. 6, 2001.

A number of attempts have been made to compensate for the movement of the lesion during the irradiation.

U.S. Pat. No. 6,725,078 (Bucholz) assigned to St. Louis University and issued Mar. 6, 2001 discloses a combined MRI and radiotherapy system which operate simultaneously but without interference so that the location of the lesion can be tracked during the radiotherapy.

U.S. Pat. No. 6,731,970 (Schlossbanner) assigned to BrainLab and issued May 4, 2004 discloses a method for breath compensation in radiation therapy, where the movement of the target volume inside the patient is detected and tracked in real time during radiation by a movement detector. The tracking is done using implanted markers and ultrasound.

U.S. Pat. No. 6,898,456 (Erbel) assigned to BrainLab and issued May 24, 2005 discloses method for determining the filling of a lung, wherein the movement of an anatomical structure which moves with breathing, or one or more points on the moving anatomical structure whose movement trajectory is highly correlated with lung filling, is detected with respect to the location of at least one anatomical structure which is not spatially affected by breathing, and wherein each distance between the structures is assigned a particular lung filling value. There is also disclosed a method for assisting in radiotherapy during movement of the radiation target due to breathing, wherein the association of lung filling values with the distance of the moving structure which is identifiable in an x-ray image and the structure which is not spatially affected by breathing is determined, the current position of the radiation target is detected on the basis of the lung filling value, and wherein radiation exposure is carried out, assisted by the known current position of the radiation target.

U.S. Pat. No. 7,265,356 (Pelizzari) assigned to University of Chicago and issued Sep. 4, 2007 discloses an image-guided radiotherapy apparatus and method in which a radiotherapy radiation source and a gamma ray photon imaging device are positioned with respect to a patient area so that a patient can be treated by a beam emitted from the radiotherapy apparatus and can have images taken by the gamma ray photon imaging device. Radiotherapy treatment and imaging can be performed substantially simultaneously and/or can be performed without moving the patient in some embodiments.

U.S. Pat. No. 7,356,112 (Brown) assigned to Elektra and issued Apr. 8, 2008 discloses that artifacts in the reconstructed volume data of cone beam CT systems can be removed by the application of respiration correlation techniques to the acquired projection images. To achieve this, the phase of the patients breathing is monitored while acquiring projection images continuously. On completion of the acquisition, projection images that have comparable breathing phases can be selected from the complete set, and these are used to reconstruct the volume data using similar techniques to those of conventional CT. This feature in the projection images can be used to control delivery of therapeutic radiation dependent on the patient's breathing cycle, to ensure that the tumor is in the correct position when the radiation is delivered.

The same company Elekta AB of Stockholm Sweden, as set out in an undated page taken from their web site, have developed a machine using CT guided radiation where CT is used to image the patient just prior to irradiation. They state that better margins can be set using motion view sequential imaging.

There are previous proposals for using MRI magnets to monitor treatment using electron beams created by a linear accelerator. The problem with this is the difficulty of combining linear accelerators and MRI. This arises because the magnetic field generated by the magnet of course interferes with the operation of the linear accelerator to an extent which cannot be readily overcome. It has however been found that relatively low field MRI units can be used with gamma radiation produced from cobalt −60. There is also a group in Edmonton who are developing a low field MRI with a LINAC.

In U.S. Pat. No. 5,735,278 (Hoult et al) issued Apr. 7, 1998, is disclosed a medical procedure where a magnet is movable relative to a patient and relative to other components of the system. The moving magnet system allows intra-operative MRI imaging to occur more easily in neurosurgery patients, and has additional applications for liver, breast, spine and cardiac surgery patients.

### SUMMARY OF THE INVENTION

It is an object of the present invention to provide an arrangement to convert the real time images that have previously been obtain with the MR modality to the real time images of the soft organ tissue during the radiation treatment with a LINAC device.

According to a first aspect of the invention there is provided a method for targeting an action on a region of interest in a body part of a body of a patient, where the body part moves within the body of the patient during functioning of the patient comprising:

using an MR imaging system to obtain at least one MR image of the body part within the patient so as to locate the region of interest within the body part;

during the action on the body part of the patient, obtaining real time ultrasound images of the body part of the patient as the body part moves within the body of the patient;

registering the MR image of the body part with the ultrasound image of the body part so as to locate the region of interest in the ultrasound image of the body part;

and using the registered images to target the action to the region of interest as the body part moves.

Preferably the ultrasound images are obtained using an ultrasound imaging probe.

Preferably the MR image of the body part is registered with the ultrasound image of the body part by imaging the probe in the MR image of the body part and including on the probe components which are located in the MR image so as to locate the probe in the MR image. However other techniques for registering the MR image with the US image are possible and do not reply on parts or components of the probe system being located in or visible in the MR image when taken. However his is a convenient way of doing the registration.

Preferably the probe carries at least one RF coil arrangement for use in obtaining the MR image and wherein the RF coil arrangement is used as a probe component for locating the probe in the MR image. However other components can also be used as the visible components and it is not essential for the RF coils to be part of or mounted on the US probe.

Preferably the body part is an organ of the body visible in the ultrasound image.

According to a second aspect of the invention there is provided a method for targeting radiation therapy on a region of interest in a body part of a body of a patient, where the body part moves within the body of the patient during the radiation therapy, the method comprising:

using an MR imaging system to obtain at least one MR image of the body part within the patient so as to locate the region of interest within the body part;

during the radiation therapy, obtaining real time ultrasound images of the body part of the patient as the body part moves within the body of the patient;

registering the MR image of the body part with the ultrasound image of the body part so as to locate the region of interest in the ultrasound image of the body part;

and using the registered images to target the radiation therapy to the region of interest as the body part moves.

Preferably the ultrasound images are obtained using an ultrasound imaging probe located adjacent or within the body of the patient.

Preferably the MR image of the body part is registered with the ultrasound image of the body part by imaging the probe in the MR image of the body part and including on the probe components which are located in the MR image so as to locate the probe in the MR image.

Preferably the position of the probe is registered with the radiation therapy by obtaining an image of the probe relative to the radiation therapy system.

Preferably the probe carries at least one RF coil arrangement for use in obtaining the MR image and wherein the RF coil arrangement is used as a probe component for locating the probe in the MR image.

Preferably the position of the probe is registered with the radiation therapy by obtaining an image of the probe relative to the radiation therapy system and by locating the RF coil in the image.

Preferably the probe carries at least one RF coil arrangement for use in obtaining the MR image.

Preferably the radiation therapy is generated by a collimated radiation source, such as a LINAC, which is rotated round the region of interest in a manner which controls the application of a required dose of radiation to the region while accommodating the shape of the region and the movement of the body part. Other sources besides a LINAC can also be used such as for example the Gamma knife which uses a Cobalt source. Thus such other radiation therapy devices can be also coexist with the MR in the presence of the ultrasound.

Preferably the radiation therapy is provided by a radiation source where the radiation source and a treatment support for the patient are located in a room shielded to prevent release of the radiation and wherein the MR imaging is carried out at a location outside the room.

According to a third aspect of the invention there is provided a method for targeting movement of a probe member into a region of interest in a body part of a body of a patient, where the body part moves within the body of the patient during the targeting of the probe member, the method comprising:

using an MR imaging system to obtain at least one MR image of the body part within the patient so as to locate the region of interest within the body part;

using an ultrasound probe movable relative to the body part to obtain real time ultrasound images of the body part of the patient as the body part moves within the body of the patient;

providing the probe member at a predetermined location relative to the ultrasound probe such that the probe member is moved in conjunction with movement of the ultrasound probe;

registering the MR image of the body part with the ultrasound images of the body part so as to locate the region of interest in the ultrasound images of the body part;

and using the registered images to target movement of the probe member to the region of interest as the body part moves.

Preferably the MR image of the body part is registered with the ultrasound image of the body part by imaging the probe in the MR image of the body part and including on the probe components which are located in the MR image so as to locate the probe in the MR image.

Preferably the ultrasound probe carries at least one RF coil arrangement for use in obtaining the MR image and wherein the RF coil arrangement is used as a probe component for locating the ultrasound probe in the MR image.

Preferably the probe member is directly mounted on the ultrasound probe to form a common probe assembly.

Preferably the common probe assembly carries at least one RF coil arrangement for use in obtaining the MR image.

Preferably the ultrasound probe is formed of a non-ferromagnetic material so as to be acceptable within the MR imaging system.

The probe member may be a biopsy probe for obtaining a sample from a region of interest in the body part, an insertion device for use in brachytherapy or for applying an interventional procedure to the body part concerned.

The invention further includes the probe assembly per se which comprises:

a probe member for movement to a body part of a patient;

an ultrasound probe for obtaining ultrasound images of the body part;

and an RF coil arrangement for use in obtaining MR images;

the probe member, the ultrasound probe and the RF coil being mounted on a common assembly for common movement.

In addition the invention includes the probe assembly for use in a radiation therapy system for treatment of a body part of a patient comprising:

an ultrasound probe for obtaining ultrasound images of the body part;

an RF coil arrangement for use in obtaining MR images of the body part;

the ultrasound probe and the RF coil being mounted on a common assembly for common movement;

the common assembly including components thereof for locating the probe in the MR image;

the common assembly including components thereof for locating the probe in the radiation therapy system.

Preferably the magnet is an annular magnet surrounding a longitudinal axis and is moved longitudinally of its axis.

The arrangement described herein correlates the outstanding high resolution MR images with the images that can be obtained in the presence of MR or LINAC. The present invention relates to the fact that a common platform for characterizing the real time motion of the soft organs, during breathing, cardiac or other functions, can be superimposed or fused on a MR CINE and ultrasound image to use for planning and treatment of diseases with radiation therapy or brachytherapy. In a further aspect, there is provided a dual 3-D ultrasound transducer with an incorporated MR radio frequency coil structure can be also use for real time tracking the navigation of the biopsy or brachytherapy device into the targeted area. By combining the superior image quality of the MR CINE and superimposing in real time the 3-D US image, the navigation of the biopsy and brachytherapy devices can yield the desired positional accuracy without guessing. By utilizing the RF coils to obtain the MR CINE image and the 3-D US transducer for US image and fusing them together the system provides real time navigation combined image with superior resolution than the US image alone.

Also since MR and LINAC are difficult to locate in the same space due to the significant ferromagnetic mass that exists on the LINAC machine and the effects that the presence of a strong magnetic field has on a radiation beam, the dual purpose 3D US transducer can act as a common reference frame to transfer real time CINE images from MR to the LINAC. Since US is compatible with LINAC, it can be used in real time during the radiation treatment to navigate the treatment based on the position of the soft organ tissue during breathing, cardiac or other motion and superimpose the data onto the MR CINE obtained with the same device utilizing the RF coil structure. In this case it is conceivable to obtain superior soft tissue real time image (fusing the US with pre-existed MR CINEs) and provide real time navigation during radiotherapy.

One more advantage of such a transducer is that during the planning session, the physicians will not to significantly expand the treatment area destroying healthy tissue during radiation, because at any instant of time they will be guided and be aware where the treatment area is during any conceivable motion of the patient's body.

The arrangement described herein may provide one or more of the following advantages:

a) The ability to perform in real time MR and US guided biopsies with significant accuracy on the position of the biopsy and brachytherapy devices in a moving tissue.

b) The ability to fuse MR and US images and utilize this information for real time monitoring of the radiation therapy on a LINAC machine, thereby reducing significantly the exposure of healthy tissue area during the radiation treatment.

c) Characterizing the motion of the soft tissue organ and relate in real time the MR images with the US images.

d) Having the US 3-D transducer act as a common reference frame between two modalities that can not exist at the same location due to safety and performance concerns, however utilizing the data from these devices to their fullest potential.

e) One MRI can be used to service more than one LINAC or can be used to service both external beam radiation therapy and brachytherapy. The systems using the LINAC and MRI combined cannot be used however for brachytherapy.

f) The combined MRI/US may be faster for real time imaging in the LINAC machine than combined systems.

g) A stand alone device which is simpler and more effective than combined systems.

Ultra sound is much more flexible than MRI but has much lower image quality. The idea of registering the MRI image to the US is to improve the image quality and retain the flexibility.

### BRIEF DESCRIPTION OF THE DRAWINGS

One embodiment of the invention will now be described in conjunction with the accompanying drawings in which:

FIG. 1 is a schematic side elevation of a radiation therapy room into which a magnet of an MRI system has been moved for imaging.

FIG. 2 is a schematic side elevation of the radiation therapy room of FIG. 1 from which the magnet of the MRI system has been removed after imaging.

FIG. 3 is a schematic illustration of an abdomen transducer with 3-D ultrasound transducer and an array of MR RF coils for use in the method of FIGS. 1 and 2.

FIG. 4 is a schematic illustration of a prostate biopsy probe with 3-D US transducer and array of MR RF coils for use in the method using the MR system of FIG. 1.

In the drawings like characters of reference indicate corresponding parts in the different figures.

### DETAILED DESCRIPTION

Reference is made to the following published applications of the present Applicant, the disclosures of which are incorporated herein by reference and to which reference should for further details of the constructions described schematically herein:

US20080039712A1 published Feb. 14, 2008 and entitled Movable Integrated Scanner for Surgical Imaging Applications;

US20090124884A1 published Feb. 14, 2008 and entitled CONTROL OF MAGNETIC FIELD HOMOGENEITY IN MOVABLE MRI SCANNING SYSTEM;

US20090306495A1 published Dec. 10, 2009 and entitled PATIENT SUPPORT TABLE FOR USE IN MAGNETIC RESONANCE IMAGING;

US20090306494A1 published Dec. 10, 2009 and entitled SYSTEM FOR MAGNETIC RESONANCE AND X-RAY IMAGING.

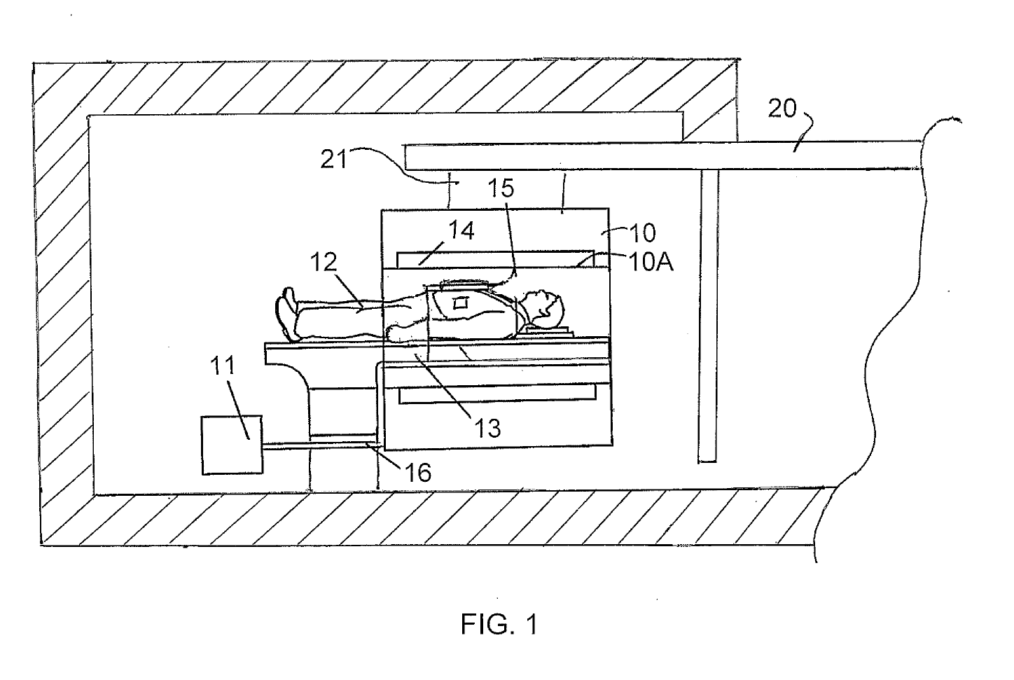

In FIG. 1 is shown schematically a magnetic resonance imaging system which includes a magnet 10 having a bore 10A into which a patient 12 can be received on a patient table 13. The system further includes an RF transmit body coil 14 which generates a RF field within the bore.

The movable magnet is carried on a rail system 20 with a support 21 suspended on the rail system. The system further includes a receive coil system generally indicated at 15 which is located at the isocenter within the bore and receives signals generated from the human body in conventional manner. An RF control system 11 acts to control the transmit body coil 14 and to receive the signals from the receive coil 15.

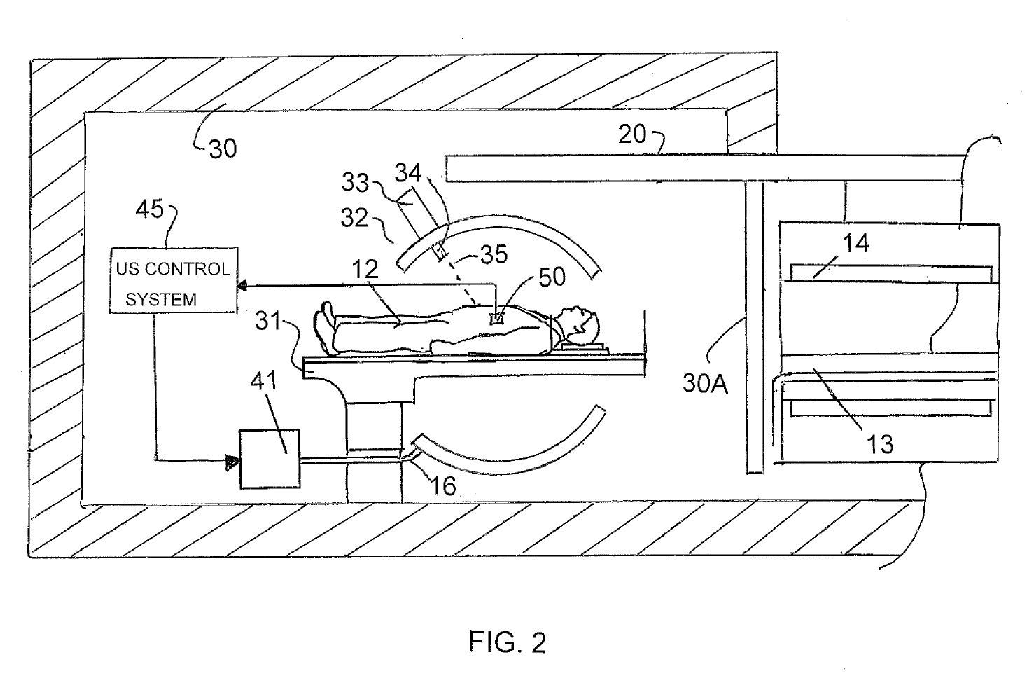

The MRI system is used in conjunction with a patient radiation therapy system shown better in FIG. 2 with the magnet 10 of the MRI system removed or with the patient moved to the radiation therapy system at a separate location. Thus the therapy system includes a bunker or room 30 within which is mounted a patient support 31 and a radiation gantry 32. The gantry carries a radiation source 33, which is in most cases a linear accelerator associated with a collimator 34 for generating a beam 35 of radiation. Systems are available for example from Siemens where the radiation system and the patient support are controlled to focus the beam onto any lesion of any shape within the patient body, bearing in mind complex shapes of lesion which are required to be radiated.

The patient having a lesion requiring radiation therapy is placed on the treatment support 31 and prepared for the radiation therapy on the treatment support.

During the initial imaging phase, the patient is located in the magnet of the MRI system. The MRI system is used while the patient is on the treatment support to obtain a series of images of the location of the lesion within the patient. These can be single images or can be sequential high-speed images.

In the treatment location, the patient is placed on the support or couch which can move such that the electron beam always irradiates the target volume. The gantry rotates such that the focus of the beam is always a relatively small volume. The table can move in three directions and this combined with the rotation focuses the treatment within a specified volume which is arranged to be as close as possible to the margins of the lesion in the patient. The goal is that this volume is the target lesion and only the target lesion. It is required that the entire target lesion receives the same maximum dose of radiation so that all cells within the targeted volume die. It is required that damage to adjacent normal tissue be minimal. Obviously, when the targeted lesion is moving the role of the MRI is to provide precise location of the lesion to that radiation unit so that it irradiates only tumor.

The radiation system includes a radiation control unit 41 which includes an electrical interface which allows control of its radiation beam over location and time.

The method for targeting radiation therapy on a region of interest or lesion in a body part of a body of a patient includes the MR system of FIG. 1 and the radiation therapy system of FIG. 2. Thus the system uses the MR imaging system to obtain at least one MR image of the body part within the patient so as to locate the region of interest within the body part.

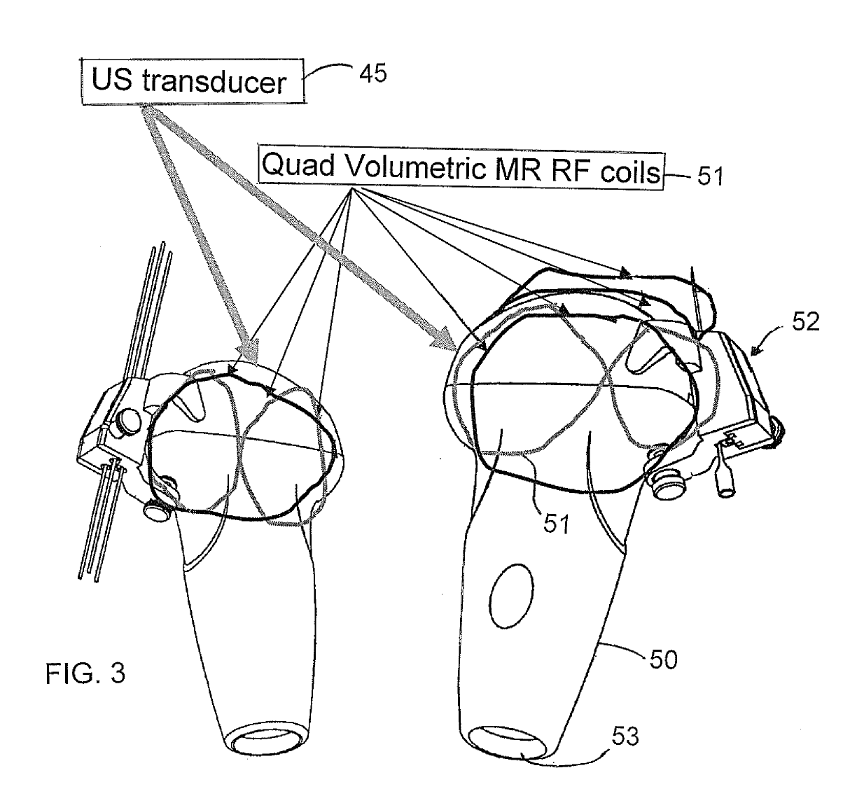

Turning now to FIG. 3 there is shown an ultrasound probe 50, which is itself of a conventional nature, on which is mounted a series of RF coil elements 51. The combination of the coil elements with the US probe forms a novel combination despite the fact that the US probe itself and the RF coil elements are both of a conventional nature. The coil elements are designed using conventional systems so as to receive the signals from the body of the patient during the MR imaging.

The coils can be phased arrays receive only in nature or can be quadrature volumetric arrays in nature. Alternatively the coils can be a combination of phased arrays as receive coil and quadrature volumetric transmit coil as a hybrid system. Persons skilled in this art know how to form such coils. The mounting of the coils on the US probe or within the body of the US probe is also within the skill of a person familiar with these products.

As the coils 51 are located at a specific or known position on the US probe, their position is automatically registered with the probe in the MR and US images. Thus the MR image provides data regarding the location of the RF coils in the mage of the body part of the patient which data automatically locates the probe 50 itself in the MR images.

During the radiation therapy, real time ultrasound images of the body part of the patient are obtained using the probe 50 as the body part moves within the body of the patient. The probe 50 can be located within the body of the patient or externally depending on the location to be treated. The probe 50 is mounted on a suitable support 52 in a manner which allows adjustment of the probe 50 and its receptor location 53.

The MR image of the body part is registered with the ultrasound image of the body part so as to locate the lesion of the body part of the patient in the ultrasound image of the body part. This is carried out at the US control system 45 which obtains the image of the body part from the probe 50 and, having the previously created MR data relating to the position of the lesion on the body part, the control system acts to superimpose the position of the lesion on the US image of the body part as that body part moves due to the movement of the body.

The registered images obtained by the control system 45 are transferred to the radiation control system so as to control the operation of the radiation system to target the radiation therapy to the lesion as the body part moves.

Thus the MR image of the body part is registered with the ultrasound image of the body part by imaging the probe in the MR image of the body part and including on the probe components which are located in the MR image so as to locate the probe in the MR image. This is preferably carried out by using the RF coil arrangement as a probe component for locating the probe in the MR image. However other MR visible markers on the probe 50 may be used to locate the probe itself in the MR image of the body part concerned. The position of the US probe 50 in the US image is of course known due to the known geometry of the US system.

The radiation therapy is generated by a collimated radiation source which is rotated round the region of interest in a manner which controls the application of a required dose of radiation to the region while accommodating the shape of the region and the movement of the body part as detected by the US probe.

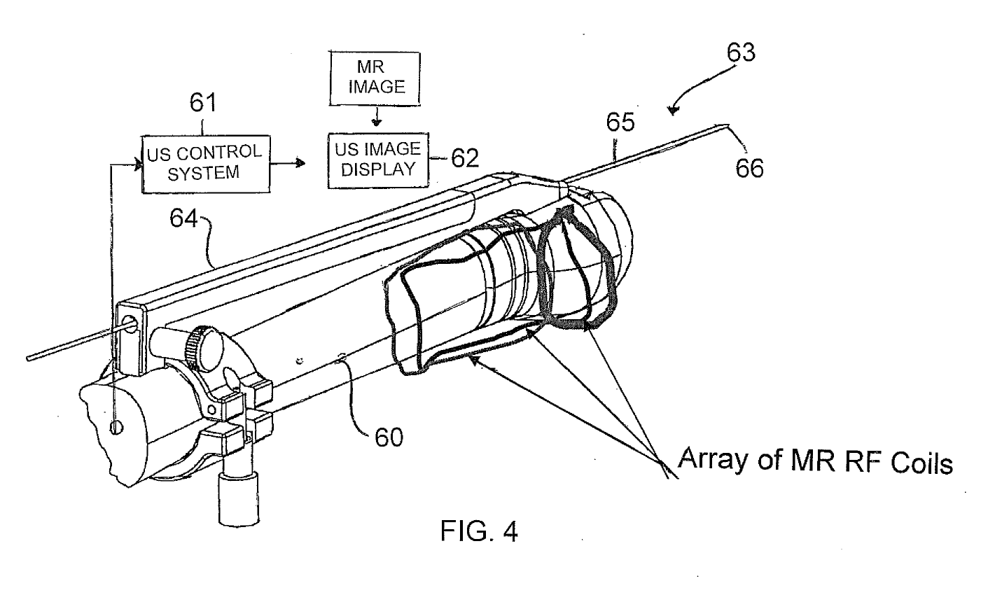

Turning now to FIG. 4, there is shown a method for targeting movement of a probe member into a region of interest in a body part of a body of a patient, where the body part moves within the body of the patient during the targeting of the probe member. The probe can be a biopsy probe as shown in FIG. 4 used to remove a targeted part of the body part of the patient or can be a probe of the type used in brachytherapy systems where the probe carries a radiation source and acts to target a region of interest of the body part of the patient to deliver that radiation source to the required location.

As described previously, the system uses an MR imaging system to obtain at least one MR image of the body part within the patient so as to locate the region of interest within the body part.

In FIG. 4, the probe is mounted on a common structure with the US probe and the RF coils, as previously described.

As explained in the previous embodiment, the coils can be phased arrays receive only in nature or can be quadrature volumetric arrays in nature. Alternatively the coils can be a combination of phased arrays as receive coil and quadrature volumetric transmit coil as a hybrid system.

The system acts to use the ultrasound probe 60 which is movable relative to the body part to obtain real time ultrasound images of the body part of the patient as the body part moves within the body of the patient. These images are obtained by the control system 61 and can be displayed on a monitor 62 for observation by the person managing the movement of the common structure 63. The probe member 64 including a needle or probe 65 with a tip 66 is mounted on the common structure 63 at a predetermined location relative to the ultrasound probe 60 such that the probe member 64 is moved in conjunction with movement of the ultrasound probe and such that the tip 66 of the probe is at a predetermined location relative to the US probe and the image obtained by the US probe and shown on the monitor 62.

The MR image of the body part previously obtained including the location of the probe in the image is registered with the real time US images of the moving body part so as to locate the region of interest or lesion to be probed in the ultrasound images of the body part as shown on the monitor 62.

In this way the person managing the movement of the probe member 64 to the required location can use the registered US and MR images to target movement of the probe member to the region of interest while observing the movement of the body part in the body.

As previously described, the MR image of the body part is registered with the ultrasound image of the body part by imaging the probe in the MR image of the body part and including on the probe components, typically but not necessarily the RF coils, which are located in the MR image so as to locate the probe in the MR image.

Thus as shown in FIG. 4, the probe member 64 is directly mounted on the ultrasound probe 60 to form a common probe assembly. In order to allow its use in the MTR imaging system, the ultrasound probe 60 and the probe member 64 are formed of a non-ferromagnetic material so as to be acceptable within the MR imaging system.

The probe member in FIG. 4 can be used as a biopsy probe for obtaining a sample from a region of interest in the body part. In this case the image taken by the US probe is arranged along the line of the probe needle 65 so as to be able to locate the tip 66 and the direction of movement of the tip in the image of the body part and the image of the region of interest in that body part.

The probe member in FIG. 4 can be used as a brachytherapy probe. In a further arrangement not shown, the probe 65 may include additional needles for transporting the various radiation sources in a more complex brachytherapy system to the required locations in the body part.

The probe member can also be arranged for applying other interventional procedures to the body part, such procedures being known to a person skilled in this art.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method for targeting an action on a region of interest in a body part of a body of a patient, where the body part moves within the body of the patient during functioning of the patient, the method comprising:
using an MR imaging system to obtain at least one MR image of the body part within the patient so as to locate the region of interest within the body part;
during the action on the body part of the patient, obtaining real time ultrasound images of the body part of the patient as the body part moves within the body of the patient;
registering the MR image of the body part with the ultrasound image of the body part so as to locate the region of interest in the ultrasound image of the body part;
and using the registered images to target the action to the region of interest as the body part moves.

2. The method according to claim 1 wherein the ultrasound images are obtained using an ultrasound imaging probe.

3. The method according to claim 2 wherein the MR image of the body part is registered with the ultrasound image of the body part by imaging the probe in the MR image of the body part and including on the probe components which are located in the MR image so as to locate the probe in the MR image.

4. The method according to claim 3 wherein the probe carries at least one RF coil arrangement for use in obtaining the MR image and wherein the RF coil arrangement is used as a probe component for locating the probe in the MR image.

5. The method according to claim 2 wherein the probe carries at least one RF coil arrangement for use in obtaining the MR image.

6. The method according to claim 1 wherein the body part is an organ of the body visible in the ultrasound image.

7. A method for targeting radiation therapy on a region of interest in a body part of a body of a patient, where the body part moves within the body of the patient during the radiation therapy, the method comprising:
using an MR imaging system to obtain at least one MR image of the body part within the patient so as to locate the region of interest within the body part;
during the radiation therapy, obtaining real time ultrasound images of the body part of the patient as the body part moves within the body of the patient;
registering the MR image of the body part with the ultrasound image of the body part so as to locate the region of interest in the ultrasound image of the body part;
and using the registered images to target the radiation therapy to the region of interest as the body part moves.

8. The method according to claim 7 wherein the ultrasound images are obtained using an ultrasound imaging probe located adjacent or within the body of the patient.

9. The method according to claim 8 wherein the MR image of the body part is registered with the ultrasound image of the body part by imaging the probe in the MR image of the body part and including on the probe components which are located in the MR image so as to locate the probe in the MR image.

10. The method according to claim 9 wherein the position of the probe is registered with the radiation therapy by obtaining an image of the probe relative to the radiation therapy system.

11. The method according to claim 9 wherein the probe carries at least one RF coil arrangement for use in obtaining the MR image and wherein the RF coil arrangement is used as a probe component for locating the probe in the MR image.

12. The method according to claim 11 wherein the position of the probe is registered with the radiation therapy by obtaining an image of the probe relative to the radiation therapy system and by locating the RF coil in the image.

13. The method according to claim 8 wherein the probe carries at least one RF coil arrangement for use in obtaining the MR image.

14. The method according to claim 7 wherein the radiation therapy is generated by a collimated radiation source which is rotated round the region of interest in a manner which controls the application of a required dose of radiation to the region while accommodating the shape of the region and the movement of the body part.

15. The method according to claim 7 wherein the radiation therapy is provided by a radiation source where the radiation source and a treatment support for the patient are located in a room shielded to prevent release of the radiation and wherein the MR imaging is carried out at a location outside the room.

16. A method for targeting movement of a probe member into a region of interest in a body part of a body of a patient, where the body part moves within the body of the patient during the targeting of the probe member, the method comprising:
using an MR imaging system to obtain at least one MR image of the body part within the patient so as to locate the region of interest within the body part;
using an ultrasound probe movable relative to the body part to obtain real time ultrasound images of the body part of the patient as the body part moves within the body of the patient;
providing the probe member at a predetermined location relative to the ultrasound probe such that the probe member is moved in conjunction with movement of the ultrasound probe;
registering the MR image of the body part with the ultrasound images of the body part so as to locate the region of interest in the ultrasound images of the body part;
and using the registered images to target movement of the probe member to the region of interest as the body part moves.

17. The method according to claim 16 wherein the MR image of the body part is registered with the ultrasound image of the body part by imaging the probe in the MR image of the body part and including on the probe components which are located in the MR image so as to locate the probe in the MR image.

18. The method according to claim 17 wherein the ultrasound probe carries at least one RF coil arrangement for use in obtaining the MR image and wherein the RF coil arrangement is used as a probe component for locating the ultrasound probe in the MR image.

19. The method according to claim 16 wherein the probe member is directly mounted on the ultrasound probe to form a common probe assembly.

20. The method according to claim 19 wherein the common probe assembly carries at least one RF coil arrangement for use in obtaining the MR image.

21. The method according to claim 16 wherein the ultrasound probe is formed of a non-ferromagnetic material so as to be acceptable within the MR imaging system.

22. The method according to claim 16 wherein the probe member is a biopsy probe for obtaining a sample from a region of interest in the body part.

23. The method according to claim 16 wherein the probe member is arranged for use in brachytherapy.

24. The method according to claim 16 wherein the probe member is arranged for applying an interventional procedure to the body part.
